(19) United States
(12) Patent Application Publication
(10) Pub. No.: US 2025/0195267 A1
(43) Pub. Date: Jun. 19, 2025

(54) METHODS OF TREATING INTRAOCULAR DISEASES BY DRUG INJECTION USING INTRAOCULAR TUBE

(71) Applicant: Masatoshi Murata, Morioka (JP)

(72) Inventor: Masatoshi Murata, Morioka (JP)

(21) Appl. No.: 18/538,326

(22) Filed: Dec. 13, 2023

(51) Int. Cl. A61F 9/00

(52) U.S. Cl. CPC A61F 9/0017; A61F 2210/009; A61F 2250/0002; A61F 2250/0068

(57) ABSTRACT

The present method provides methods of treating an intraocular diseases comprising: (a) a physician places an internally-facing end of a tube for drug injection into an intraocular space of a patient; and (b) under a guidance of the physician, a drug can be remotely injected into the intraocular space through the externally-facing end (outside of the external canthus) of the tube.

### TECHNICAL FIELD

Embodiments described herein generally relate to a method of treating an intraocular disease by self-injection of a therapeutically effective dose of drug solution using an intraocular tube that connects the outside of external canthus to the intraocular space.

### BACKGROUND OF THE INVENTION

For the treatment of intraocular diseases, routes of administration of the drug include topical administration via the ocular surface by eye drop, systemic administration via the systemic circulating bloodstream by injection, infusion and the like, and direct administration into the anterior chamber and/or vitreous body. However, it is difficult to deliver a minimum effective dose of drug to the intraocular space (IOS) over an extended period.

For example, topical administration by eye drop is prone to insufficient penetration into the IOS due to lacrimation and corneal impermeability. Systemic administration requires large doses of drug and may result in various side effects, such as hypertension. Anterior chamber and/or intravitreal injections need to be repeated to maintain the therapeutic level of drug and are prone to causing pain for the patient, and carry potential risks such as glaucoma, cataract, retinal detachment, and endophthalmitis (Geroski D H, Edelhauser H F. “Drug delivery for posterior segment eye disease.” Invest Ophthalmol Vis Sci. 2000; 41:961-964).

The present inventor has focused on tube insertion, which has been used in a variety of medical fields, including ophthalmology. The present inventor has found that, by inserting a tube into the eye for injecting a drug into an affected area, a therapeutically effective dose of the drug can be repeatedly delivered to the IOS over a long period of time with fewer risks such as a burden on the patient and side effects, and the drug solution can be remotely injected into the IOS, that is, remote from the operational setting of tube placement, e.g., by manual operation by a patient or caregiver, or by an automated or wireless injector.

### BRIEF SUMMARY OF THE INVENTION

That is, an object of the present invention is to provide a method of treating intraocular disease by remote injection of a therapeutically effective dose of drug solution using intraocular tube that connects outside of external canthus to intraocular space.

According to the present invention, the following method of treating intraocular disease can be provided.

In one embodiment, the present invention provides a method of treating an intraocular disease comprising:
(a1) a physician places an internally-facing end of a tube for drug injection into an anterior chamber of a patient; and
(b1) under a guidance of the physician, remotely injecting a drug into the anterior chamber through the placed tube, wherein the step (a1) comprises
(a1-1) incising a conjunctiva and piercing a tube insertion needle into a corneal limbus,
(a1-2) advancing a tip of the tube insertion needle into the anterior chamber,
(a1-3) inserting the internally-facing end of the tube into the anterior chamber from an inlet of the tip of the tube insertion needle, and
(a1-4) placing the internally-facing end of the tube into the anterior chamber and pulling out the tube insertion needle.

In another embodiment, the present invention provides a method of treating an intraocular disease comprising:
(a2) a physician places an internally-facing end of a tube for drug injection into a vitreous body of a patient; and
(b2) under a guidance of the physician, remotely injecting a drug into the vitreous body through the placed tube, wherein
the step (a2) comprises
(a2-1) incising a conjunctiva and piercing a tube insertion needle into a sclera near a corneal limbus,
(a2-2) advancing a tip of the tube insertion needle into the vitreous body,
(a2-3) inserting the internally-facing end of the tube into the vitreous body from an inlet of the tip of the tube insertion needle, and
(a2-4) placing the internally-facing end of the tube into the vitreous body and pulling out the tube insertion needle.

In yet another embodiment, the present invention provides a method of treating an intraocular disease comprising:
(a3) a physician places an internally-facing end of a tube for drug injection into a choroid of a patient; and
(b3) under a guidance of the physician, remotely injecting a drug into the choroid through the placed tube, wherein
the step (a3) comprises
(a3-1) incising a conjunctiva, and piercing a tube insertion needle between the conjunctiva and a sclera,
(a3-2) advancing a tip of the tube insertion needle to a vicinity of an optic disc along a surface of the sclera, placing a lens over a cornea so that a fundus can be observed, and observing the tip of the tube insertion needle that can be observed as a white raised portion of the sclera while pressing the sclera using the tip of the tube insertion needle and observing the fundus through the lens,
(a3-3) moving the tip of the tube insertion needle to determine an appropriate insertion position of the tube in the vicinity of the optic disc,
(a3-4) piercing the tip of the tube insertion needle diagonally into the sclera, advancing the tip of the tube insertion needle into the choroid, and inserting the internally-facing end of the tube into the choroid through an inlet of the tip of the tube insertion needle,
(a3-5) placing the internally-facing end of the tube into the choroid and pulling out the tube insertion needle, and
(a3-6) incising the skin of the external canthus, guiding an externally-facing end of the tube through the subconjunctiva and subcutaneous, and exposing the externally-facing end under the incised skin.

In any of the methods described herein, the placement of the tube can further comprise incising the skin of the external canthus, guiding the externally-facing end of the tube through the subconjunctiva and subcutaneous, and exposing the externally-facing end of the tube under the incised skin.

In any of the methods described herein, the methods can further comprise implanting a magnet in an intraocular pocket to attract a drug formulation comprising a magnetetic substance toward a target tissue. For example, a magnet can be implanted in a corneal pocket, an equatorial scleral pocket, or a posterior polar scleral pocket.

In any of the methods described herein, the method can further comprise inserting and securing a catheter of a reservoir at the externally-facing (exposed) end of the tube, and sealing a tank of the reservoir with a material pierceable by a drug injection needle.

In any of the methods described herein, the drug is capable of being injected through the intraocular tube in a setting that is remote from the operational setting where the intraocular tube was inserted. In one embodiment, the drug is injected manually, by the patient themselves, by a layperson caregiver, or by a medically trained provider. In one embodiment, the drug is self-injected by the patient. In another embodiment, the drug can be injected automatically using a wireless injector. In yet another embodiment, the drug can be continuously administered using a micropump.

The methods described herein can be used to deliver a drug to an intraocular target, such as the anterior chamber, the vitreous body, and/or the choroid. Drug delivery to the anterior chamber can be used to treat intraocular diseases including, but not limited to: glaucoma, cataract, keratitis, and anterior uveitis. Drug delivery to the vitreous body can be used to treat intraocular diseases including, but not limited to: glaucoma, vitreous opacity, uveitis, diabetic retinopathy, retinal vein occlusion, macular degeneration, and retinitis pigmentosa. Drug delivery to the choroid can be used to treat intraocular diseases including, but not limited to: glaucoma, diabetic retinopathy, macular degeneration, retinal vein occlusion, uveitis, retinochoroidal degeneration, and scleritis.

These methods of treatment described herein allow a therapeutically effective dose of the drug to be repeatedly delivered to the IOS over a long period of time with fewer risks such as a burden on the patient and side effects.

### BRIEF DESCRIPTION OF THE DRAWINGS

FIG. 1 is a schematic diagram showing the structure of a human eye.

FIG. 2 is a schematic diagram illustrating an example of a tube insertion system.

FIG. 3 shows a photograph of a tube insertion system.

FIG. 4 is a schematic diagram illustrating a state in which a conjunctiva is incised, and a tube insertion needle is inserted between the conjunctiva and the sclera when a tube is inserted into the choroid (the conjunctiva is omitted).

FIG. 5 is a schematic diagram illustrating a state in which the tip of a tube insertion needle is advanced along the surface of the sclera when a tube is inserted into the choroid.

FIG. 6 is a schematic diagram illustrating a state in which the tip of a tube insertion needle is advanced to the vicinity of the optic disc along the surface of the sclera, and a lens is placed over the cornea so that the fundus can be observed when a tube is inserted into the choroid.

FIG. 7 is an enlarged schematic diagram illustrating a state in which the tip of a tube insertion needle is diagonally inserted into the sclera when a tube is inserted into the choroid.

FIG. 8 is an enlarged schematic diagram illustrating a state in which the tip of a tube insertion needle is diagonally inserted into the choroid when a tube is inserted into the choroid.

FIG. 9 is an enlarged schematic diagram illustrating a state in which the tip of a tube insertion needle is advanced within the choroid when a tube is inserted into the choroid.

FIG. 10 is an enlarged schematic diagram illustrating a state in which one end of the tube for drug injection is inserted into the choroid through an inlet of the tip of the tube insertion needle when a tube is inserted into the choroid.

FIG. 11 is a schematic diagram illustrating an example of the step of placing one end of the tube for drug injection into the choroid when a tube is inserted into the choroid.

FIG. 12A is a photograph of a tube (arrow) inserted into the anterior chamber of the left eye of a rabbit. FIG. 12B is a photograph showing a reservoir (long arrow) connected to an end of a tube (short arrow) on the conjunctival side.

FIG. 13A is a photograph of a tube (arrow) inserted into the anterior vitreous body of the right eye of a rabbit. FIG. 13B is a photograph showing a reservoir connected to an end of a tube on the conjunctival side.

FIG. 14A is a photograph of a tube (arrow) inserted into the choroid in the vicinity of the optic disc of the left eye of a rabbit. FIG. 14B is a photograph of a 20 G tube insertion needle (arrow) pierced to guide the end of a tube on the conjunctival side under the conjunctiva and under the skin. FIG. 14C is a photograph showing a reservoir connected to an end of a tube on the conjunctival side. FIG. 14D is a photograph of injecting a drug into an implanted reservoir (short arrow) using a 24 G drug injection needle (long arrow) and a 1 cc syringe.

FIG. 15A is a photograph of a tube (arrow) inserted into the choroid in the vicinity of the optic disc of the left eye of a rabbit. FIG. 15B shows a tube (arrow) found in the choroidal space by optical coherence tomography (OCT) examination, corresponds to the location of the tube in FIG. 15A.

FIG. 16A shows a tube (arrow) inserted into the anterior chamber (Cor: cornea, Iri: iris, scale bar: 250 μm), and no abnormalities were found in the surrounding tissues. FIG. 16B shows the tube (arrow) is still present in the choroid, and although the photoreceptor outer segment and outer nuclear layer were disrupted, no abnormalities were found in the surrounding tissue (Ret: retina, Cho: choroid, scale bar: 250 μm).

FIG. 17A shows a photograph of injecting ICG into the anterior chamber of the left eye of a rabbit using a wireless injector (arrow). FIG. 17B is a photograph of ICG (arrow) injected into the anterior chamber.

FIG. 18A is a photograph of a micropump (arrow) located under the skin near the external canthus of the left eye of a rabbit. FIG. 18B is a photograph of ICG (arrow) injected into the anterior chamber. FIG. 18C is a photograph of an implanted micropump where the wounds are sutured with 6-0 silk and covered with a transparent dressing.

FIG. 19A shows iron nanoparticles (short arrow) stained with Prussian blue in the inner surface of porcine cornea near the intracorneal implanted site (long arrow) of neodymium magnet. FIG. 19B shows iron nanoparticles (short arrow) located in the retinal surface near the intra-scleral implanted site (long arrow) of neodymium magnet. FIG. 19C shows stained iron nanoparticles (short arrow) in the choroidal surface adjoining the intra-scleral implanted site (long arrow) of neodymium magnet.

### DETAILED DESCRIPTION OF THE INVENTION

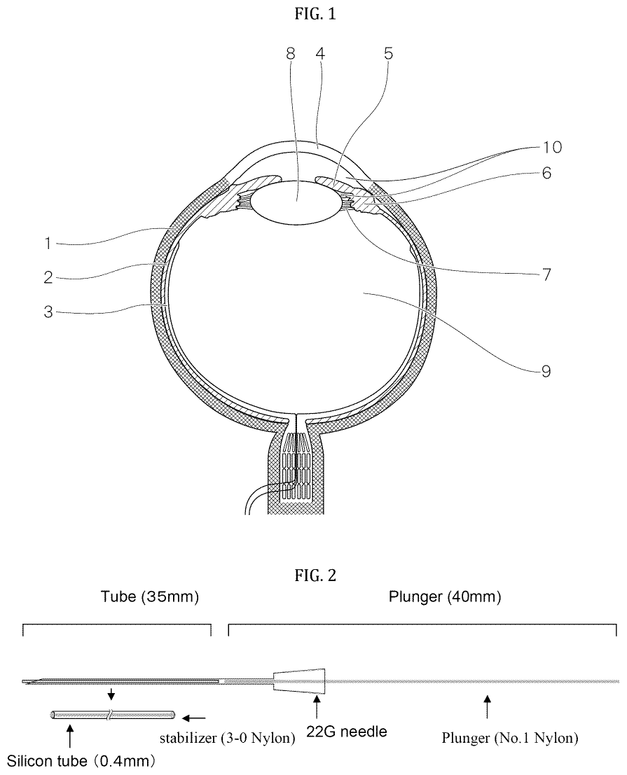

Hereinafter, embodiments of the method for treating an intraocular disease according to the present invention will be described below in detail with reference to Drawings. The structure of the eye is described below with reference to FIG. 1. The upper side in FIG. 1, i.e., the side where the cornea is present, is referred to as the anterior side in the structure of the eye, and the lower side in FIG. 1, i.e., the side where the optic nerve is present, is referred to as the posterior side in the structure of the eye.

### Structures of Eye

An eye includes an eyeball, adnexa, and optic nerves. As illustrated in FIG. 1, the eyeball includes outer wall members such as a sclera 1, a choroid 2, a retina 3, a cornea 4, an iris 5, a ciliary body 6, and a zonule of Zinn 7, and content members such as a crystalline lens 8, a vitreous body 9, and an aqueous humor 10. The adnexa include a lacrimal apparatus, a conjunctiva, and the like.

The portion surrounded by the cornea 4 and the crystalline lens 8 is called an anterior chamber and a posterior chamber, with the iris 5 as the boundary. The location where the cornea 4 and the iris 5 meet is referred to as an angle.

The sclera 1 is a milky white, hard membrane. The anterior end of the sclera 1 is connected to the cornea. The sclera 1 allows light to pass through to only a small extent, and prevents the entry of unnecessary light into the eyeball. The sclera 1 thus protects the inside of the eyeball. The sclera 1 has a thickness of about 1.0 mm to about 1.5 mm in an area in which the sclera 1 forms the rear wall of the eyeball, and has a minimum thickness of about 0.5 mm in an area in which the eye muscle adheres to the sclera 1.

The choroid 2 is a thin blackish brown membrane that is situated between the sclera 1 (that is situated on the outer side) and the retina 3 (that is situated on the inner side). A number of blood vessels are present in the choroid 2, and supply nutrients to the outer layer of the retina in which blood vessels are not present. The thickness of the choroid 2 is about 0.2 mm to about 0.3 mm.

The retina 3 is a thin membrane that includes ten layers. The retina 3 has a thickness of about 0.3 mm to about 0.4 mm in the center area, and has a thickness of about 0.15 mm in the peripheral area. Two types of photoreceptor cells (rod cells and cones) are present in the retina 3. Important cells such as ganglion cells and Müller cells and a number of blood vessels are also present in the retina 3. A macular area in which a number of cones are present is situated at the center of the rear part of the retina 3. The macular area is part of the eyeball that has the best visual acuity.

The vitreous body 9 is a colorless and transparent gel with which most of the interior of the eyeball is filled. Water accounts for 99% of the vitreous body 9. The vitreous body 9 is situated behind the crystalline lens. The vitreous body 9 is bonded to the retina 3 in a deep area of the eyeball, but most of the vitreous body 9 comes in light contact with the retina 3.

### Intraocular Disease

In this specification, intraocular disease includes all ocular diseases that occur in the intraocular space (IOS). Intraocular diseases include, for example, ocular diseases that occur in the anterior chamber, angle, crystalline lens, vitreous body, retina, choroid, and sclera.

Examples of the intraocular disease include, but are not limited to, for example, glaucoma, cataracts, keratitis, anterior uveitis, uveitis, diabetic retinopathy, retinal vein occlusion, macular degeneration, retinitis pigmentosa, vitreous opacity, retinal vein occlusion, retinochoroidal degeneration, scleritis, and the like.

Next, methods for treating an intraocular disease will be described.

### 1. First Method for Treating Intraocular Disease

A method for treating an intraocular disease according to the first aspect of the present invention (hereinafter, also referred to as “first treating method”) contains the steps of:
(a1) a physician places an internally-facing end of a tube for drug injection into an anterior chamber of a patient; and
(b1) under a guidance of the physician, the patient remotely injects a drug into the anterior chamber through the placed tube.
the step (a1) comprises
(a1-1) incising a conjunctiva and piercing a tube insertion needle into a corneal limbus,
(a1-2) advancing a tip of the tube insertion needle into the anterior chamber,
(a1-3) inserting the internally-facing end of the tube into the anterior chamber from an inlet of the tip of the tube insertion needle, and
(a1-4) placing the internally-facing end of the tube into the anterior chamber and pulling out the tube insertion needle.

According to the first treating method, an internally-facing end of a tube is placed in the anterior chamber (AC), and a therapeutically effective dose of the drug can be repeatedly delivered to the anterior chamber over a long period of time directly with fewer risks such as a burden on the patient and side effects, and the drug solution can be remotely injected, e.g., self-injected into the anterior chamber by the patient's own operation.

This allows for the treatment of intraocular diseases that occur near the anterior chamber. Examples of the intraocular disease that occurs near the anterior chamber include, but are not limited to, for example, glaucoma, cataract, keratitis, anterior uveitis, and the like.

It should be noted that the first treating method is not limited to the specific matters described in the following embodiments and Examples as long as the method contains incising a conjunctiva and inserting a tube insertion needle into a corneal limbus, advancing a tip of the tube insertion needle into the anterior chamber, and inserting an internally-facing end of the tube into the anterior chamber from an inlet of the tip of the tube insertion needle. In addition, in some Examples described below, a rabbit is used as an object to be treated, but all methods of the present invention can be applied to other research animals and/or clinical subjects including veterinary and human subjects in the same manner.

a) Tube insertion system

For the tube insertion system and the placed intraocular tube, tubes and needles such as those used in various medical fields including ophthalmology can be used. For example, a silicon tube can be used.

In the method of inserting an intraocular tube for drug delivery, a tube insertion system configured to insert a 3-0 nylon (e.g., length: 35 mm) into a 0.4 mm silicone insertion tube (e.g., length: 35 mm), insert the 0.4 mm silicone tube into a 22 G needle, and insert a No. 1 nylon (e.g., length: 40 mm) into the needle from the rear of the needle, as shown in FIG. 2, can be suitably used. The No. 1 nylon thread (that serves as a plunger) is used to push the tube toward the outside through the tip of the tube insertion needle. Also, the 3-0 nylon serves as an inner stabilizer to support the tube and insert the tube into the anterior chamber.

The dimensions of the tube, the tube insertion needle, the plunger, and the inner stabilizer are not limited to the above combinations, and can be freely selected as long as the purpose of inserting the tube into the target site is achieved.

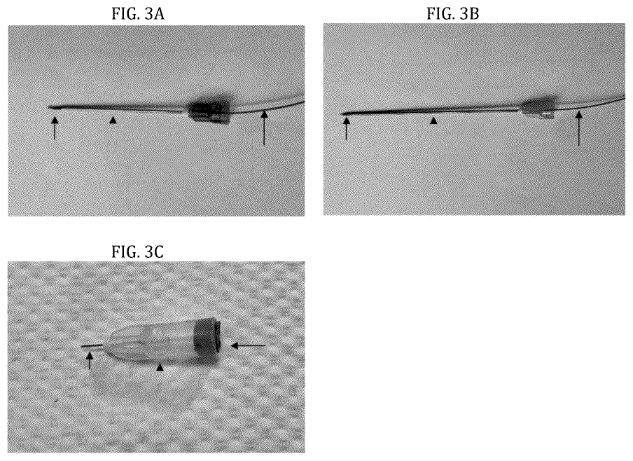

As an example, in FIG. 3A, an intraocular tube insertion system composed of a silicone tube (short arrow), a stabilizer (not visible in the photograph), a plunger (long arrow), and a 22 G needle (arrowhead) is shown. Specific examples of the use are described in “(i) Tube insertion into anterior chamber” and “(ii) Tube insertion into anterior vitreous body” of Examples.

As another example, in FIG. 3B, an intraocular tube insertion system composed of a silicone tube (short arrow), a stabilizer (not visible in the photograph), a plunger (long arrow), and a 21 G cathelin needle (arrowhead) is shown. Specific examples of the use are described in “(iii) Tube insertion into the choroid” of the Examples.

In the following, an intraocular tube insertion system composed of a silicone tube (short arrow), a stabilizer (not visible in photographs), a plunger (long arrow), and a 22 G tube insertion needle (arrowhead) as shown in FIG. 3A is used to explanation.

The silicone tube may have a reservoir, such as shown in FIG. 3C, connected to the end opposite the end that is inserted into the IOS. The reservoir can be chosen freely as long as the purpose of delivering fluid from the tank through the catheter is achieved. For example, a reservoir having a tank, a catheter for delivering liquid from the tank, and a septum for sealing the tank can be used.

As an example, in FIG. 3C, a photograph of a reservoir with a 32 G drug injection needle sealed with a rubber cap. The body portion of the drug injection needle corresponds to the tank, the needle portion of the drug injection needle corresponds to the catheter, and the rubber cap portion corresponds to the septum. The septum is preferably made of a material that can be pierced with a drug injection needle. Specific examples of the material include butyl rubber, silicone rubber, and the like.

Note that the reservoir shown in FIG. 3C is an example. As described above, the shape, the material, and other features of the reservoir are not limited to this example, as long as the purpose of delivering the liquid from the tank through the catheter is achieved. As an example, the reservoir can be made of a biocompatible material. The reservoir can be provided with a portion for threading to fix reservoir site. The inside of the tank and/or catheter can be coated with heparin to prevent the formation of blood clots.

In this specification, the method of inserting a tube is explained using the configuration of the tube insertion system shown in FIG. 2.

It is preferable to use nylon as a material for forming the plunger, for example. When a wire is used as the plunger, it may be difficult to push the plunger along the curved tube insertion needle since the plunger may be caught within the curved tube insertion needle due to too high a hardness. When a silk thread is used as the plunger, the plunger can be pushed along the curved tube insertion needle, but it may be difficult to push the tube forward due to too high a softness. Since nylon has moderate hardness and elasticity, nylon is suitable as the material for forming the plunger. Note that the material of the plunger is not limited to nylon, and any material having the same hardness can be suitably used as in the case of nylon.

The plunger can be moved manually, for example by pushing the rear end of the plunger with a finger. The injector can also be used to move the plunger wirelessly. Once the tube is inserted, the injector can also be used to inject drug solution wirelessly. For example, a pen-type electric dispenser manufactured by ICOMES LAB Co. Ltd. may suitably be used as the injector.

It is preferable that the tube insertion needle have a size as small as possible from the viewpoint of invasiveness. From such viewpoint, it is preferable that the outer diameter of the main body of the tube insertion needle is 1.0 mm or smaller, 0.9 mm or smaller, or 0.8 mm or smaller. The inner diameter of the main body of the tube insertion needle is preferably 0.7 mm or smaller, 0.6 mm or smaller, or 0.5 mm or smaller. The size of the needle is preferably 21 G, 22 G, 23 G, 24 G, or 25 G, and it is more preferable if a tube insertion needle having a smaller size can be used.

b) Tube Insertion into Anterior Chamber

The step (a1) is a step in which a physician places one end of a tube for drug injection into an anterior chamber and contains the steps (a1-1) to (a1-4) in this order.

In the step (a1-1), the conjunctiva that covers the eyeball of the subject is incised, and a tube insertion needle is pierced into a corneal limbus.

In the step (a1-2), the tip of the tube insertion needle inserted into the corneal limbus in the step (a1-1) is advanced into the anterior chamber.

In the step (a1-3), one end of the tube for drug injection is inserted into the anterior chamber through an inlet of the tip of the tube insertion needle advanced into the anterior chamber. The tube can be inserted, for example, by pushing the plunger of the tube insertion system described above.

In the step (a1-4), the tube insertion needle, the plunger, and the stabilizer are withdrawn while one internally-facing end of the tube is left in place in the anterior chamber. This leaves only the tube for drug injection, with one internally-facing end placed in the anterior chamber. In addition, the externally-facing end of the tube for drug injection exits outside the AC from the site where the tube insertion needle was inserted in the step (a1-1).

In one embodiment, the step (a1) contains, prior to the step (a1-1) or after the step (a1-4),
(a1-0) placing an implant having a magnet (hereinafter referred to as a “magnet implant”) in a corneal pocket.

This enables drug delivery to transport drugs to specific sites using magnetic force. Specifically, by injecting a drug delivery carrier holding a drug and a magnetic substance through a tube inserted into the anterior chamber in the step (a1), the magnetic substance in the carrier is attracted to the magnet in the corneal pocket, thereby increasing the drug concentration locally at the target site in the anterior chamber, improving the therapeutic effect at the target site, and reducing side effects at sites other than the target site. The type and strength of the magnet, the insertion site of the magnet implant, or the type of the carrier are not particularly limited as long as the purpose of attracting the magnetic substance in the carrier to the target site is achieved.

As the magnet contained in the implant, a permanent magnet can be used, for example. It is preferable to use a neodymium magnet because of the strength of the magnetic force and the ease of availability.

As a carrier for drug delivery, a carrier which is generally used in the field of drug delivery can be used without any particular limitation as long as it can hold a drug and a magnetic substance. For example, liposomes, lactic acid-glycolic acid copolymers, and the like can be used.

As the magnetic substance in the drug formulation, for example, iron nanoparticles, iron oxide nanoparticles, or the like can be used.

The method of placing the magnet implant in the corneal pocket may be any method as long as the pocket is made by incising a target position on the cornea, and the magnet implant is placed in the pocket, and is not limited to the specific matters described in the following embodiments and Examples.

In one embodiment, the step (a1) contains, after the step (a1-4),
(a1-5) incising the skin of the external canthus, guiding the other end of the tube through the subconjunctiva and subcutaneous, and exposing the end under the incised skin.

This makes it easier to accurately and stably inject the drug through the tube.

In one embodiment, the step (a1) contains, after the step (a1-5),
(a1-6) inserting and securing a catheter of a reservoir at the externally-facing end of the tube, and sealing a tank of the reservoir with a material pierceable by a drug injection needle.

This makes it easier to stably and accurately inject the drug through the reservoir over a long period of time.

c) Drug Injection into Anterior Chamber

In the step (b1), under a guidance of the physician, the patient injects a drug into the anterior chamber through the tube with one end placed in the anterior chamber.

As a result, drug solution can be remotely injected, e.g., self-injected into the anterior chamber by the patient's own operation. Performed under the guidance of the physician via telemedicine, the patient can receive treatment from the comfort of their own home.

The drug may be injected directly from the outer body end of the tube for drug injection or through a reservoir connected to the outer body end of the tube.

In one embodiment, the drug injection is performed automatically using a wireless injector. The function and shape of the wireless injector are not particularly limited as long as the purpose of automatically injecting the drug is achieved.

In one embodiment, the drug injection is performed continuously using a micropump. The function and shape of the micropump are not particularly limited as long as the purpose of continuously injecting the drug is achieved.

The use of these devices makes it easier to inject the drug stably and accurately over a long period of time. As a result, it is expected that the burden on the eyeball is reduced, and the concentration of the drug is stabilized.

### 2. Second Method for Treating Intraocular Disease

A method for treating an intraocular disease according to the second aspect of the present invention (hereinafter, also referred to as “second treating method”) contains the steps of:
(a2) a physician places an internally-facing end of a tube for drug injection into a vitreous body of a patient; and
(b2) under a guidance of the physician, the patient remotely injects a drug into the vitreous body through the placed tube.

The step (a2) comprises:
(a2-1) incising a conjunctiva and piercing a tube insertion needle into a sclera near a corneal limbus,
(a2-2) advancing a tip of the tube insertion needle into the vitreous body,
(a2-3) inserting the internally-facing end of the tube into the vitreous body from an inlet of the tip of the tube insertion needle, and
(a2-4) placing the internally-facing end of the tube into the vitreous body and pulling out the tube insertion needle.

According to the second treating method, an internally-facing end of a tube is placed in the vitreous body, and a therapeutically effective dose of the drug can be repeatedly delivered to the vitreous body over a long period of time directly with fewer risks such as a burden on the patient and side effects, and the drug solution can be remotely injected, e.g., self-injected into the vitreous body by the patient's own operation.

This allows for the treatment of intraocular diseases that occur near the vitreous body. Examples of the intraocular disease that occurs near the vitreous body include, but are not limited to, for example, glaucoma, vitreous opacity, uveitis, diabetic retinopathy, retinal vein occlusion, macular degeneration, retinitis pigmentosa, and the like.

It should be noted that the second treating method is not limited to the specific matters described in the following embodiments and Examples as long as the method contains incising a conjunctiva and inserting an injection needle into a sclera near a corneal limbus, advancing a tip of the injection needle into the vitreous body, and inserting one end of the tube into the vitreous body from an inlet of the tip of the injection needle. In addition, in some Examples described below, a rabbit is used as an object to be treated, but all methods of the present invention can be applied to other research animals and/or clinical subjects including veterinary and human subjects in the same manner.

a) Tube insertion system

With respect to the tube insertion system, the matters described in the first treating method can be applied. Similar to the first treating method, the method of inserting the tube will be described using the configuration of the tube insertion system of FIG. 2.

As a specific tube insertion system, an intraocular tube insertion system composed of a silicone tube (short arrow), a stabilizer (not visible in photographs), a plunger (long arrow), and a 22 G injection needle (arrowhead) as shown in FIG. 3A is used to explanation.

b) Tube Insertion into Vitreous Body

The step (a2) is a step in which a physician places an internally-facing end of a tube for drug injection into a vitreous body of a patient, and contains the steps (a2-1) to (a2-4) in this order.

In the step (a2-1), the conjunctiva that covers the eyeball of the subject is incised, and a tube insertion needle is pierced into a sclera near a corneal limbus.

In the step (a2-2), the tip of the tube insertion needle pierced into the sclera near the corneal limbus in the step (a2-1) is advanced into the vitreous body.

In the step (a2-3), an internally-facing end of the tube is inserted into the vitreous body from an inlet of the tip of the tube insertion needle. The tube can be inserted, for example, by pushing the plunger of the tube insertion system described above.

In the step (a2-4), the tube insertion needle, the plunger, and the stabilizer are withdrawn while the internally-facing end of the tube is left in place in the vitreous body. This leaves only the tube for drug injection, with the internally-facing end placed in the vitreous body. In addition, the externally-facing end of the tube for drug injection is left outside the anterior vitreous body (AV) from the site where the tube insertion needle was inserted in the step (a2-1).

In one embodiment, the step (a2) contains, prior to the step (a2-1) or after the step (a2-4),
(a2-0) placing an implant having a magnet (hereinafter referred to as a “magnet implant”) in an equatorial scleral pocket.

This enables drug delivery to transport drugs to specific sites using magnetic force. Specifically, by injecting a drug delivery carrier holding a drug and a magnetic substance through a tube inserted into the vitreous body in the step (a2), the magnetic substance in the carrier is attracted to the magnet in the equatorial scleral pocket, thereby increasing the drug concentration locally at the target site in the vitreous body, improving the therapeutic effect at the target site, and reducing side effects at sites other than the target site.

With respect to the magnetic implant and drug delivery carrier, the matters described in the first treating method can be applied.

For example, the magnetic implant may be placed in the equatorial scleral pocket by the following method:

First, under an operating microscope, a conjunctival flap was made by a fornix-based conjunctival incision.

Next, a scleral incision of the half thickness was made 3 mm parallel to the corneal limbus and at an equator site.

Subsequently, a 25-gauge MVR blade was moved ahead in parallel to the surface of the sclera and also moved in left and right directions, thereby creating a pocket made of an incision of 3 mm×3 mm.

Finally, a magnet implant is inserted into this pocket.

In one embodiment, the step (a2) contains, after the step (a2-4),
(a2-5) incising the skin of the external canthus, guiding the externally-facing end of the tube through the subconjunctiva and subcutaneous, and exposing the externally-facing end under the incised skin.

This makes it easier to accurately and stably inject the drug through the tube.

In one embodiment, the step (a2) contains, after the step (a2-5),
(a2-6) inserting and securing a catheter of a reservoir at the externally-facing end of the tube, and sealing a tank of the reservoir with a material pierceable by a drug injection needle.

This makes it easier to stably and accurately inject the drug through the reservoir over a long period of time.

c) Drug Injection into Vitreous Body

In the step (b2), under the guidance of a physician, the patient can manually inject a drug into the vitreous body through the tube with an internally-facing end placed in the vitreous body.

As a result, drug solution can be remotely injected, e.g., self-injected into the vitreous body by the patient's own operation. Performed under the guidance of the physician via telemedicine, the patient can receive treatment from the comfort of their own home.

The drug may be injected directly from the externally-facing end of the tube for drug injection or through a reservoir connected to the externally-facing end of the tube.

In one embodiment, the drug injection is performed automatically using a wireless injector. The function and shape of the wireless injector are not particularly limited as long as the purpose of automatically injecting the drug is achieved.

In one embodiment, the drug injection is performed continuously using a micropump. The function and shape of the micropump are not particularly limited as long as the purpose of continuously injecting the drug is achieved.

The use of these devices makes it easier to inject the drug stably and accurately over a long period of time. As a result, it is expected that the burden on the eyeball is reduced, and the concentration of the drug is stabilized.

### 3. Third Method for Treating Intraocular Disease

A method of treating an intraocular disease according to a third aspect of the present invention (hereinafter, also referred to as “third method of treatment”) contains the steps of:
(a3) a physician places an internally-facing end of a tube for drug injection into a choroid of a patient; and
(b3) under a guidance of the physician, the patient remotely injects a drug into the choroid through the placed tube.
the step (a3) comprises:
(a3-1) incising a conjunctiva, and piercing a tube insertion needle between the conjunctiva and a sclera,
(a3-2) advancing a tip of the tube insertion needle to a vicinity of an optic disc along a surface of the sclera, placing a lens over a cornea so that a fundus can be observed, and observing the tip of the tube insertion needle that can be observed as a white raised portion of the sclera while pressing the sclera using the tip of the tube insertion needle and observing the fundus through the lens,
(a3-3) moving the tip of the tube insertion needle to determine an appropriate insertion position of the tube in the vicinity of the optic disc,
(a3-4) piercing the tip of the tube insertion needle diagonally into the sclera, advancing the tip of the tube insertion needle into the choroid, and inserting the internally-facing end of the tube into the choroid through an inlet of the tip of the tube insertion needle,
(a3-5) placing the internally-facing end of the tube into the choroid and pulling out the tube insertion needle, and
(a3-6) incising the skin of the external canthus, guiding the externally-facing end of the tube through the subconjunctiva and subcutaneous, and exposing the externally-facing end under the incised skin.

According to the third treating method, an internally-facing end of the intraocular tube is placed in the choroid in the vicinity of the optic disc, and a therapeutically effective dose of the drug can be repeatedly delivered into the choroid over a long period of time directly with fewer risks such as a burden on the patient and side effects, and the drug solution can be remotely injected, e.g., self-injected into the choroid by the patient's own operation.

This allows for the treatment of intraocular diseases such as posterior segment eye diseases that occur in the vicinity of the optic disc. Examples of the intraocular disease that occurs in the vicinity of the optic disc include, but are not limited to, for example, glaucoma, diabetic retinopathy, macular degeneration, retinal vein occlusion, uveitis, retinochoroidal degeneration, scleritis, and the like.

It should be noted that the third treating method is not limited to the specific matters described in the following embodiments and Examples as long as the method contains incising a conjunctiva, and inserting a tube insertion needle between the conjunctiva and a sclera, advancing a tip of the tube insertion needle to a vicinity of an optic disc along a surface of the sclera, placing a lens over a cornea so that a fundus can be observed, and observing the tip of the tube insertion needle that can be observed as a white raised portion of the sclera while pressing the sclera using the tip of the tube insertion needle and observing the fundus through the lens, moving the tip of the tube insertion needle to determine an appropriate insertion position of the tube in the vicinity of the optic disc, piercing the tip of the tube insertion needle diagonally into the sclera, advancing the tip of the tube insertion needle into the choroid, and inserting the internally-facing end of the tube into the choroid through an inlet of the tip of the tube insertion needle, placing the internally-facing end of the tube into the choroid and pulling out the tube insertion needle, and incising the skin of the external canthus, guiding the externally-facing end of the tube through the subconjunctiva and subcutaneous, and exposing the externally-facing end under the incised skin. In addition, in some Examples described below, a rabbit is used as an object to be treated, but all methods of the present invention can be applied to other research animals and/or clinical subjects including veterinary and human subjects in the same manner.

### a) Tube Insertion System

With respect to the tube insertion system, the matters described in the first treating method can be applied. Similar to the first treating method, the method of inserting the tube will be described using the configuration of the tube insertion system of FIG. 2, except that a silicone tube (length: 55 mm) and a 21 G cathelin needle are used in place of a silicone tube (length: 35 mm) and a 22 G needle, respectively.

As a specific tube insertion system, an intraocular tube insertion system composed of a silicone tube (0.4 mm outer diameter, 55 mm length; short arrow), a stabilizer (3-0 nylon resin; not visible in photographs), a plunger (No. 1 nylon resin; long arrow), and a 21 G cathelin needle (arrowhead) as shown in FIG. 3B is used to explanation.

b) Insertion of Tube into Choroid

A method of inserting the tube for drug injection in the step (a3) will be described with reference to the Drawings. The step (a3) is a step in which a physician places an internally-facing end of a tube for drug injection into a choroid of a patient, and contains the steps (a3-1) to (a3-6) in this order.

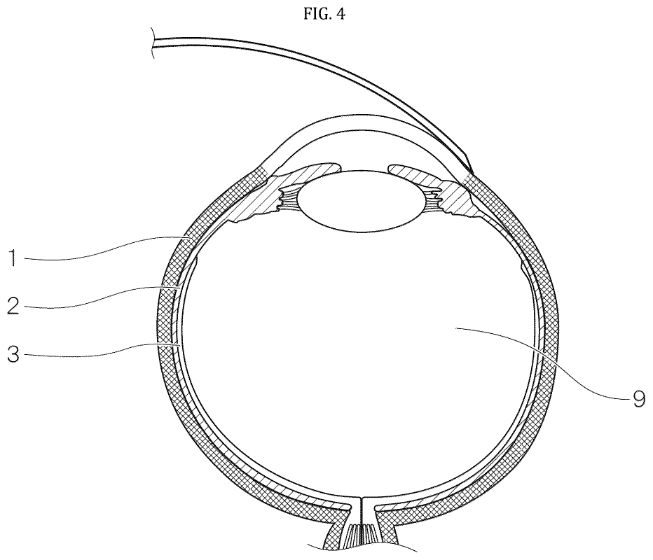

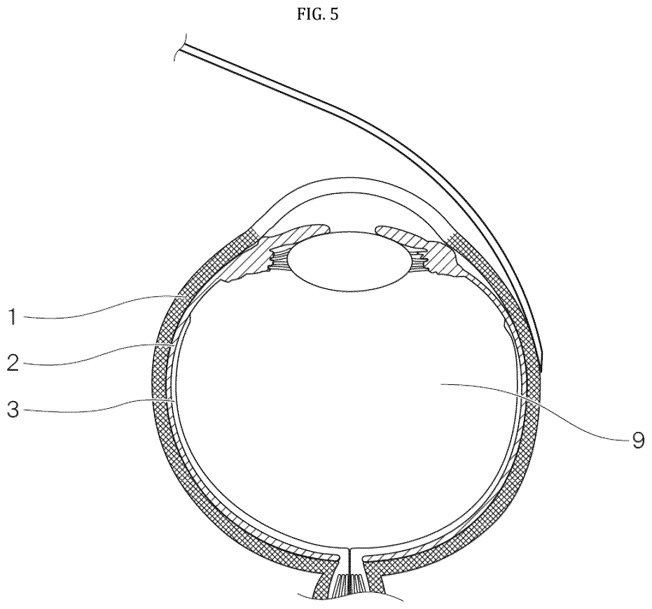

In the step (a3-1), the conjunctiva that covers the eyeball of the subject is incised, and a tube insertion needle is inserted between the conjunctiva and sclera. As illustrated in FIGS. 4 and 5, the conjunctiva that covers the eyeball is incised, and a tube insertion needle is inserted between the conjunctiva and the sclera.

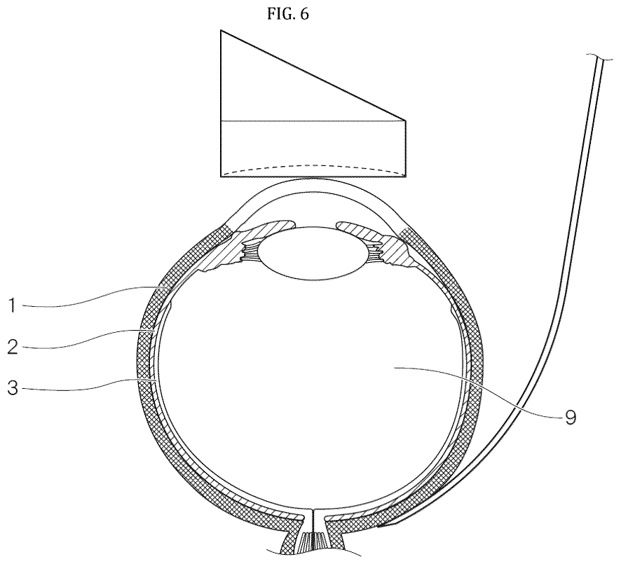

In the step (a3-2), as shown in FIG. 6, the tip of the tube insertion needle that inserted between the conjunctiva and the sclera in the step (a3-1) is advanced to a vicinity of an optic disc along a surface of the sclera, and a lens is placed over a cornea so that a fundus can be observed, and the tip of the tube insertion needle that can be observed as a white raised portion of the sclera while pressing the sclera using the tip of the tube insertion needle is checked with observing the fundus through the lens.

In the step (a3-3), the tip of the tube insertion needle is moved to determine an appropriate insertion position of the tube in the vicinity of the optic disc.

At this time, the state of the sclera is observed through the retinal side with a lens while pressing the sclera using the tip of the tube insertion needle, and the surface of the sclera that is contiguous to an area of the choroid in the vicinity of the optic disc in which the number of blood vessels is small and a large vessel is not present, is determined to be the insertion position of the tube.

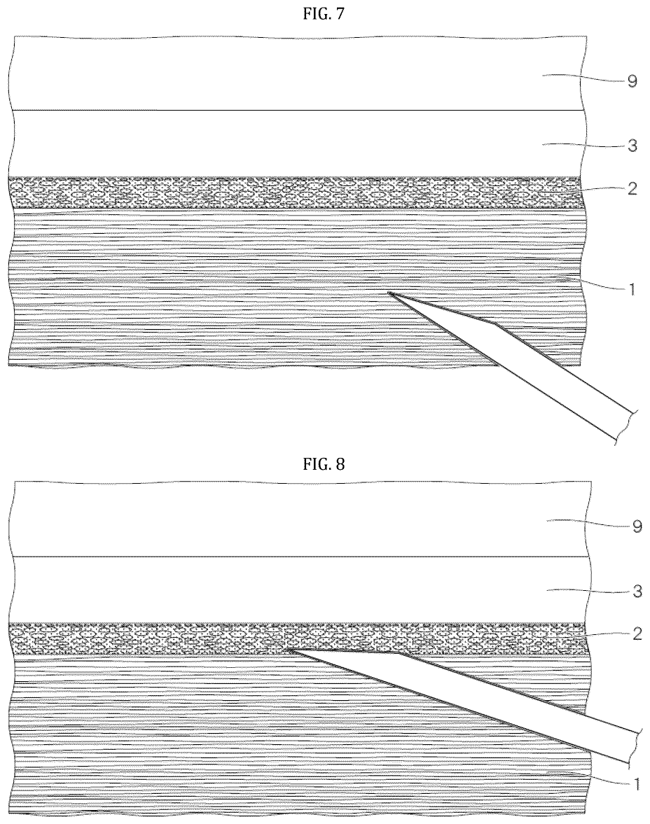

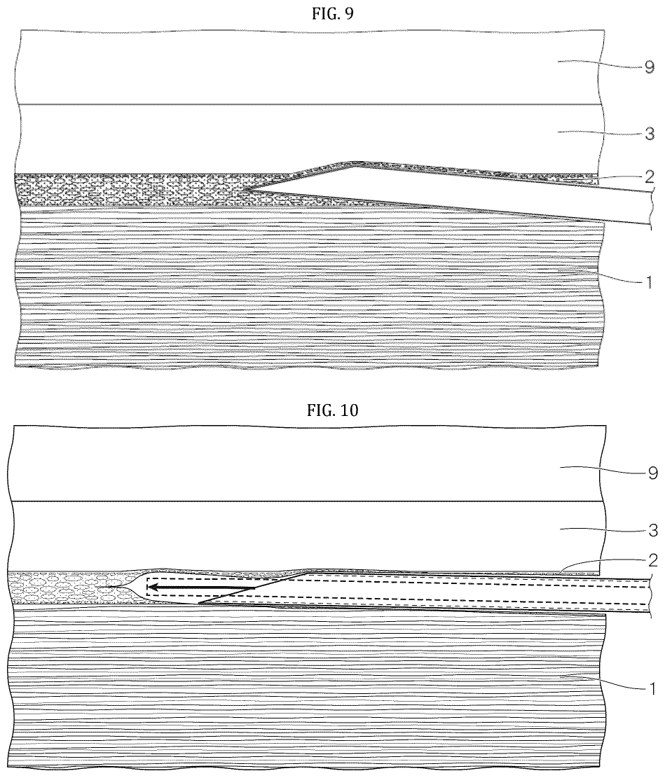

In the step (a3-4), as illustrated in FIG. 7, the tip of the tube insertion needle is diagonally pierced into the sclera. As illustrated in FIGS. 8 and 9, the tip of the tube insertion needle is then advanced into the choroid.

The insertion of the tip of the tube insertion needle is confirmed by observing the fundus through the lens. It is determined that the tip of the tube insertion needle has been inserted into the choroid when a state in which the tip of the tube insertion needle is observed through a thick membrane (i.e., an area of the sclera pressed by the tip of the tube insertion needle is observed to be a white area) has changed to a state in which the tip of the tube insertion needle is observed through a thin membrane (i.e., the tip of the tube insertion needle is clearly observed under the retina having a small thickness) when observed through the lens.

Specifically, since the sclera allows light to pass through to only a small extent, the tip of the tube insertion needle is situated under or within the sclera in a state in which the tip of the tube insertion needle is observed through a thick membrane. On the other hand, the tip of the tube insertion needle is situated under the retina and over the sclera in a state in which the tip of the tube insertion needle is observed through a thin membrane. Accordingly, it can be determined that the tip of the tube insertion needle has been inserted into the choroid when the tip of the tube insertion needle is observed through a thin membrane.

When the tip of the tube insertion needle has been inserted into the choroid, the tip of the tube insertion needle is advanced within the choroid parallel to the choroid. Specifically, the tip of the tube insertion needle is moved parallel to (in the tangential direction with respect to) the layer that forms the choroid.

The tip of the tube insertion needle is advanced within the choroid parallel to the choroid until it is observed through the lens that the inlet of the tip of the tube insertion needle has been inserted into the choroid. It is determined that the tip opening of the tube insertion needle has been inserted into the choroid when the tip opening of the tube insertion needle can be observed through a thin membrane.

Then, as illustrated in FIG. 10, the internally-facing end of the tube is inserted into the choroid through an inlet of the tip of the tube insertion needle.

In the step (a3-5), the internally-facing end of the tube is placed in the choroid and the tube insertion needle is pulled out.

In the step (a3-6), the skin of the external canthus is incised, the externally-facing end (the side not placed in the choroid, outer body end) of the tube is guided through the subconjunctiva and subcutaneous, and the externally-facing end is exposed under the incised skin.

As a result, it is possible to safely and repeatedly inject an appropriate dose of drug solution into the choroid from the externally-facing end of the tube exposed outside the skin in accordance with the condition of the disease in the vicinity of the optic disc.

Thus, according to the step (a3) of the third treating method, the tube can be inserted into the choroid in the vicinity of the optic disc, as illustrated in FIG. 10, without performing intraocular surgical procedure.

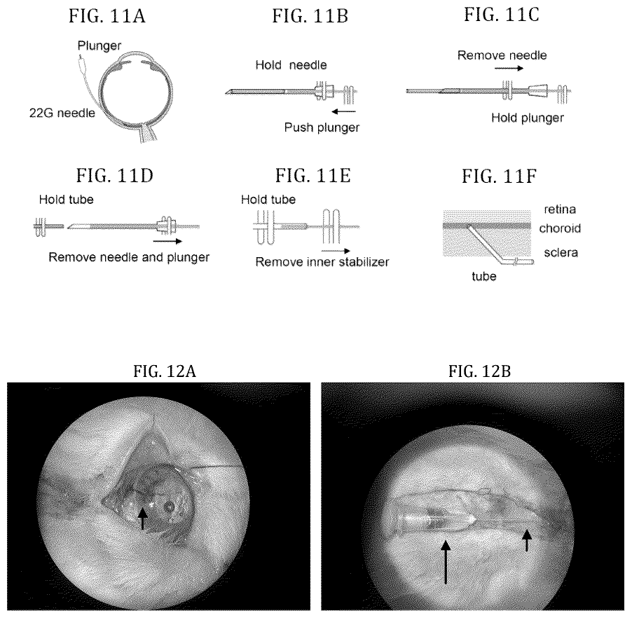

FIG. 11 schematically shows an example of an inserting step of the tube.

In the inserting step of the tube, the tip of the tube insertion needle is advanced in parallel within the choroid (FIG. 11A); and the inlet of the tube insertion needle is inserted into the choroid, and then the tube (and 3-0 nylon) is inserted into the choroid from the tip of the tube insertion needle by pressing the plunger (No. 1 nylon) with the right hand while fixing the tube insertion needle with the left hand, for example, using tweezers or the like (FIG. 11B).

The tube insertion needle is removed from the choroid and the sclera while fixing the plunger (No. 1 nylon) so that the tube (and the 3-0 nylon thread) remains in the choroid (FIG. 11C). The tube insertion needle is removed from the tube together with the No. 1 nylon while fixing the tube so that the tube (and the 3-0 nylon thread) remains in the choroid (FIG. 11D).

The tube is then fixed to withdraw the 3-0 nylon as an inner stabilizer from the tube (FIG. 11E), and only the tube is placed in the choroid (FIG. 11F). As a result, internally-facing end of the tube opens within the choroid, and the externally-facing end of the tube opens in the vicinity of the incised conjunctiva. The internally-facing end of the tube is inserted into the choroid in a state in which the main body of the tube extends between the conjunctiva and the sclera from the vicinity of the incised conjunctiva, and passes through the sclera in the vicinity of the optic disc.

In one embodiment, the step (a3) contains, prior to the step (a3-1) or after the step (a3-6),
(a3-0) placing an implant having a magnet (hereinafter referred to as a “magnet implant”) in a posterior polar scleral pocket.

This enables drug delivery to transport drugs to specific sites using magnetic force. Specifically, by injecting a drug delivery carrier holding a drug and a magnetic substance through a tube inserted into the choroid in the step (a3), the magnetic substance in the carrier is attracted to the magnet in the posterior polar scleral pocket, thereby raising the drug concentration locally at the target site in the choroid, improving the therapeutic effect at the target site, and reducing side effects at sites other than the target site.

With respect to the magnetic implant and drug delivery carrier, the matters described in the first treating method can be applied.

For example, the magnetic implant may be placed in the posterior polar scleral pocket by the following method.

First, under an operating microscope, a conjunctival flap was made by a fornix-based conjunctival incision.

Next, a scleral incision of the half thickness was made 3 mm parallel to the corneal limbus and at a posterior polar site.

Subsequently, a 25-gauge MVR blade was moved ahead in parallel to the surface of the sclera and also moved in left and right directions, thereby creating a pocket made of an incision of 3 mm×3 mm.

Finally, a magnet implant is inserted into this pocket.

In one embodiment, the step (a3) contains, after the step (a3-6),
(a3-7) inserting and securing a catheter of a reservoir at the externally-facing end of the tube, and sealing a tank of the reservoir with a material pierceable by a drug injection needle.

This makes it easier to stably and accurately inject the drug through the reservoir over a long period of time.

c) Drug Injection into Choroid

In the step (b3), under the guidance of a physician, the patient injects a drug into the choroid through the tube with the internally-facing end placed in the choroid.

As a result, the drug solution can be remotely injected, e.g., self-injected into the choroid by the patient's own operation. Performed under the guidance of the physician via telemedicine, the patient can receive treatment from the comfort of their own home.

The drug may be injected directly from the outer body end of the tube for drug injection or through a reservoir connected to the outer body end of the tube.

In one embodiment, the drug injection is performed automatically using a wireless injector. The function and shape of the wireless injector are not particularly limited as long as the purpose of automatically injecting the drug is achieved.

In one embodiment, the drug injection is performed continuously using a micropump. The function and shape of the micropump are not particularly limited as long as the purpose of continuously injecting the drug is achieved.

The use of these devices makes it easier to inject the drug stably and accurately over a long period of time. As a result, it is expected that the burden on the eyeball is reduced, and the concentration of the drug is stabilized.

### EXAMPLES

Hereinafter, the present invention will be described in detail with reference to specific examples, but the present invention is not limited to the following Examples.

### Example 1: Fabrication of Tube Insertion System

A silicon tube (0.4 mm outer diameter, 0.3 mm inner diameter, 35 to 55 mm length, manufactured by ARAM Corporation), an inner stabilizer (3-0 nylon resin, manufactured by Akiyama-seisakusyo. Co., Ltd.), a plunger (No. 1 nylon resin, manufactured by Alfresa Pharma Corporation), and a 22 G injection needle or 21 G cathelin needle (manufactured by Terumo Corporation) were used to fabricate an intraocular tube insertion system as shown in FIGS. 2 and 3. A rubber-capped 32 G injection needle (manufactured by Terumo Corporation) (FIG. 3C) was connected thereto and used as a reservoir.

### Example 2: Insertion of Tube into IOS

All experiments were conducted in accordance with ARVO Statement for the Use of Animals in Ophthalmic and Vision Research and the Guidelines for Experimental Animals at Iwate Medical University. Three-month-old Japanese white rabbits (purchased from KITAYAMA LABES CO., LTD.) weighing 2 to 2.5 kg were used.

Rabbits were anesthetized with ketamine hydrochloride (24 mg/kg) and xylazine hydrochloride (6 mg/kg). The eye surface was then anesthetized with topical instillation of 2% xylocaine. The pupil was dilated with topical 0.5% tropicamide.

(i) Insertion of Tube into Anterior Chamber (AC)

First, a conjunctiva was incised to expose a sclera. Next, a tip of the 22 G tube insertion needle of the tube insertion system was then pierced into a corneal limbus and the tip of the tube insertion needle was advanced into an anterior chamber. The plunger was then pushed to insert one end of the drug injection tube into the anterior chamber.

Thereafter, the tube insertion needle, the plunger, and the stabilizer were removed, respectively, and the tube placed in the anterior chamber was observed by slit-lamp examination (FIG. 12A).

(ii) Insertion of Tube into Anterior Vitreous Body (AV)

First, a conjunctiva was incised to expose a sclera. A tip of the 22 G tube insertion needle of the tube insertion system was then pierced into the sclera at a position 2 mm from the corneal limbus, and the tip of the tube insertion needle was advanced into a vitreous body. The plunger was then pushed to insert one end of the drug injection tube into the vitreous body.

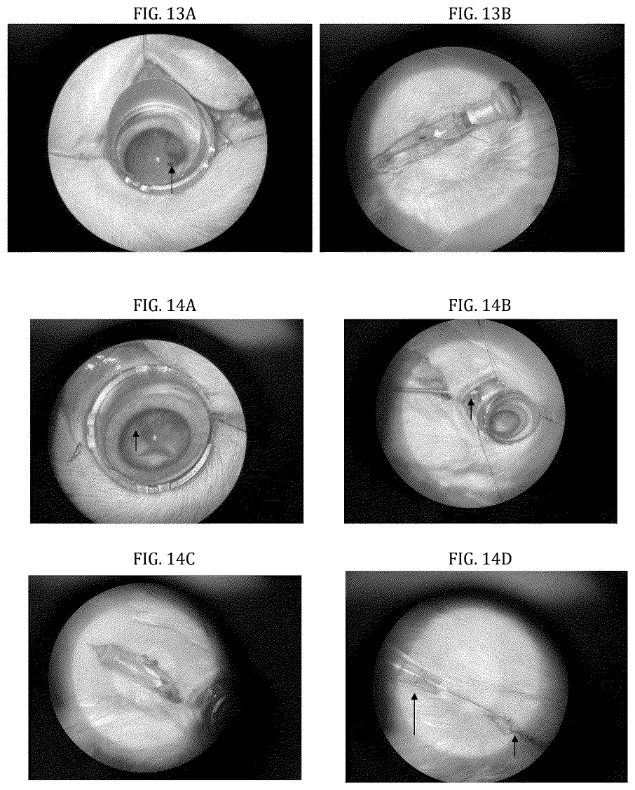

Thereafter, the tube insertion system, including the tube insertion needle, the plunger, and the stabilizer, was removed, respectively, and the tube placed in the vitreous body was observed by ophthalmoscopy (FIG. 13A).

(iii) Insertion of Tube into Choroid

First, a conjunctiva was incised 2 mm from the inferior limbus to expose a sclera. A 21 G cathelin needle was then inserted into a subconjunctiva (FIGS. 6 and 11A) and the sclera was pressed to confirm the insertion site near an optic disc. The tip was pierced shallowly into the sclera and advanced parallel to the choroid at half the choroid thickness (FIGS. 7 to 9). The plunger was then pushed and the tube was inserted into the choroid until the tip of the tube reached near the optic disc. Thereafter, the 21 G cathelin needle, the plunger, and the stabilizer were removed, respectively, and the tube placed in the vicinity of the optic disc was observed by fundus examination (FIG. 14A).

Most rabbits had mild choroidal hemorrhages, which resolved spontaneously within 1 week.

### Example 3: Implantation of Reservoir

The skin of the external canthus of the eyes was incised in all rabbits in which the tube was inserted into the AC, the AV, or the choroid in the (2) above. The conjunctival end of the tube was induced 5 mm from the outside of external canthus using a 20 G injection needle as a guide through the subconjunctiva and subcutaneous (FIG. 14B), and exposed under the incision skin. A 32 G injection needle was then connected (FIGS. 12B, 13B, and 14C). Then, the entire of the main body of the 32 G injection needle other than the opening was implanted (FIG. 14D). The opening of the main body of the injection needle was sealed with a rubber cap. The rubber cap was pierced with a 24 G injection needle connected to a 1 cc syringe, and the drug was repeatedly injected into IOS through the intraocular tube (FIG. 14D).

### Example 4: Clinico-Pathological Evaluation

To evaluate possible adverse effects of the intraocular tube insertion, treated eyes were examined by slit lamp examination and ophthalmoscopy at days 7 and 14. Eyes with tubes inserted into the choroid were examined by optical coherence tomography (OCT) on day 14. Then these eyes were enucleated after euthanasia on day 14 and evaluated by histopathology.

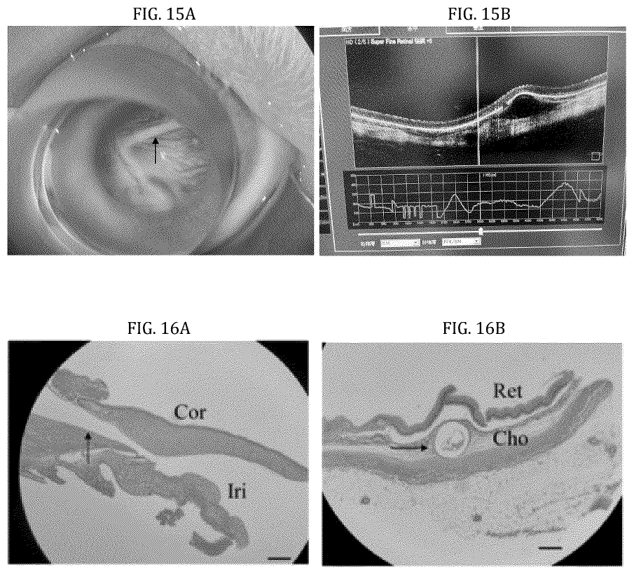

In all experiments, the conjunctiva showed no significant infection in the slit lamp examination and ophthalmoscopy, and the cornea, anterior chamber, and crystalline lens were clear during the observation period. In AC and AV tube eyes, the surrounding tissue showed no significant inflammation. The choroidal tube was still observed at 2 weeks and the retina inferior to the posterior pole around the tube site showed no abnormalities (FIG. 15A). OCT showed the tube was present in choroidal space, and there was no abnormality in the surrounding tissue of the tube (FIG. 15B).

In the pathological examination of the AC tube eye, there was no abnormality in the cornea, sclera, and iris (FIG. 16A). In the AV tube eye, the abnormality was not recognized in the surrounding tissue. The choroidal tube was still found at the insertion site without shifting. In the retina, the area corresponding to the position of the tube showed damage to the photoreceptor outer segment and outer nuclear layer. The retina except the tube site, showed no abnormalities (FIG. 16B).

### Example 5: DEX Level Measurement in Aqueous Humor and Retina

Rabbit eyes in which the AC tube is inserted were received DEX (dexamethasone, 10 μg/10 μL) by a general administration (4 eyes), a topical eye drop (4 eyes), and an anterior chamber injection (4 eyes). Before injecting DEX into the anterior chamber through the tube, approximately 20 μL of aqueous humor was aspirated through cornea limbus. As a result, the internal pressure of the anterior chamber can be reduced, thereby making the anterior chamber space to inject the drug. All eyes were aspirated aqueous humor at 1 hour after the DEX administration. DEX level in the aqueous humor was determined by liquid chromatography/mass spectrometry/mass spectrometry (LC/MS/MS) using hybrid triple quadrupole/linear ion trap mass spectrometers (AB SCIEX 3200 QTRAP, manufactured by AB Sciex Pte. Ltd.).

The AV and choroidal tube insertion eyes (4 eyes each) were injected DEX (10 μg/10 μL). Before injecting DEX, approximately 50 μL of the intravitreal fluid was aspirated through the pars plana. As a result, the internal pressure of the vitreous body can be reduced, thereby making space at the vitreous body or choroidal space to inject the drug. At 3 and 24 hours, the eye injected DEX was enucleated, and removed posterior-inferior retina. Concretely, the enucleated eye was cut by razor to expose the inferior retina. A retinal incision was made 5 mm parallel to the optic disc at inferior site, and a quadrate sample, consisting of an incision of 5×5 mm, was created. Then these samples were used to examine the DEX levels using LC/MS/MS.

The DEX concentrations of aqueous humors in eye drop and general administration groups were below the detection limit. The DEX concentration in AC tube group showed 2.24+2.77 ng/ml. The level of the DEX in AC tube group at 1 hour significantly higher than that of other groups (1 hour; p=0.014<0.05), suggesting that AC tube DEX delivery system significantly increases the anterior chamber DEX level compared with topical eye drop and general administration.

In AV and choroidal tubes, retinal DEX levels were examined at 3 and 24 hours after the DEX injection, because it has been reported that most of DEX in vitreous injection eye was released to the vitreous body within 24 hours. The DEX levels of inferior retinas in AV tube eyes at 3 and 24 hours were 328.5+24.2, and 0.7+0.5 ng/g, respectively. The DEX levels of inferior retinas in choroidal tube eyes at 3 and 24 hours were 701.0+244.9, and 2.7+3.4 ng/g, respectively.

DEX levels in the AV and choroidal tube eyes at 3 hours showed the effective dose in the inferior retina portion, because 0.15 to 4.0 μg/ml of DEX is known to be the effective dose for inhibition of inflammation. This indicates that the DEX injection through AV and choroidal tubes effectively delivers the DEX into the posterior polar retina.

### Example 6: Evaluation of Automatic Injection Using a Wireless Injector Through AC Tube

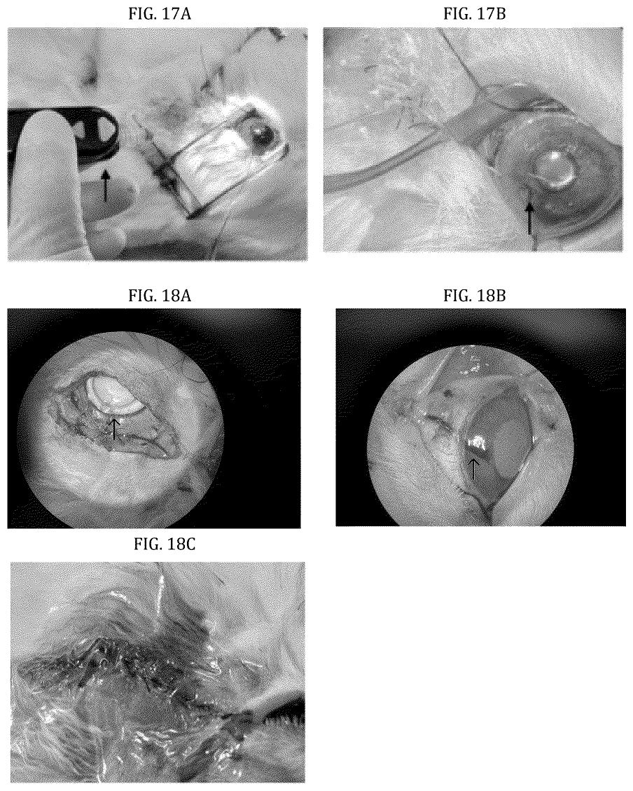

To evaluate automated injection system, we studied whether or not a wireless injector is effective to deliver indocyanine green (ICG) into the IOS automatically. The amount of ICG was determined using the application (“Tofuty,” manufactured by ICOMES LAB Co. Ltd.) installed on the computer. The data on the amount of ICG was sent to a wireless injector by bluetooth. The wireless injector was then connected to the external canthus side of the tube, and three eyes were injected the ICG solution (5 μg/10 μL: 0.05%) into the AC (FIG. 17).

ICG was automatically injected into the AC by using the wireless injector (FIG. 17B). There was no abnormality in the injected eyes using slit lamp examination. Thus, a wireless injector was shown to be useful in automatically injecting a drug into IOS through a tube.

### Example 7: Evaluation of Continuous Injection Using a Micro-Pump Through AC Tube

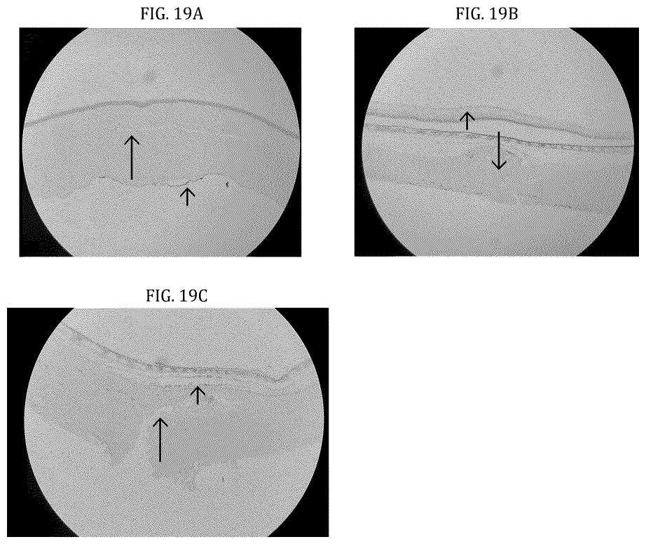

To evaluate continuous injection system, we studied whether or not a micropump is effective to deliver the ICG into the IOS continuously. A micropump (SMP-310R, manufactured by PRIMETECH CORPORATION) was connected to the AC tube, and ICG solution (5 μg/10 μL) was injected into three eyes (FIG. 18).

ICG was continuously injected into the AC using the micropump (FIG. 18B). There was no abnormality in the injected eyes using slit lamp examination. Thus, a micropump system was shown to be useful in automatically injecting a drug into IOS through a tube.

### Example 8: Assessment of Implantation of Neodymium Magnet for Maintaining Drug Level in IOS

To evaluate the maintenance of drug levels in IOS, we examined whether neodymium magnetic implants were effective in causing drug retention in IOS. A scleral or cornea incision was made in enucleated pig eye. A blade was moved in left and right directions, thereby creating a pocket having an incision of 3 mm×3 mm. Then the neodymium magnet was inserted into this pocket. After that, iron nanoparticles (20 nm iron oxide nanoparticles, manufactured by Cytodiagnostics Inc.) were dropped on the cornea, retina, and choroid, and examined histologically 1 hour later.

Histology showed iron nanoparticles stick to cornea, retina and choroid corresponding to the neodymium magnet implant site (FIG. 19). In no neodymium eyes, there were no iron particles in cornea, retina, and choroid.

Although only some exemplary embodiments and/or examples of this invention have been described in detail above, those skilled in the art will readily appreciate that many modifications are possible in the exemplary embodiments and/or examples without materially departing from the novel teachings and advantages of this invention. Accordingly, all such modifications are intended to be included within the scope of this invention.

The documents described in the specification are incorporated by reference in their entirety.

### DESCRIPTION OF SYMBOLS

1 Sclera
2 Choroid
3 Retina
4 Cornea
5 Iris
6 Ciliary body
7 Zonule of zinn
8 Crystalline lens
9 Vitreous body
10 Aqueous humor

What is claimed is:

1. A method of treating an intraocular disease comprising:
(a1) a physician places an internally-facing end of a tube for drug injection into an anterior chamber of a patient; and
(b1) under a guidance of the physician, remotely injecting a drug into the anterior chamber through the placed tube, wherein
the step (a1) comprises
(a1-1) incising a conjunctiva and piercing a tube insertion needle into a corneal limbus,
(a1-2) advancing a tip of the tube insertion needle into the anterior chamber,
(a1-3) inserting the internally-facing end of the tube into the anterior chamber from an inlet of the tip of the tube insertion needle, and
(a1-4) placing the internally-facing end of the tube into the anterior chamber and pulling out the tube insertion needle.

2. The method of treating according to claim 1, wherein the step (a1) comprises, prior to the step (a1-1) or after the step (a1-4),
(a1-0) placing an implant having a magnet in a corneal pocket.

3. The method of treating according to claim 1, wherein the step (a1) comprises, after the step (a1-4),
(a1-5) incising the skin of the external canthus, guiding an externally-facing end of the tube through the subconjunctiva and subcutaneous, and exposing the externally-facing end under the incised skin.

4. The method of treating according to claim 3, wherein the step (a1) comprises, after the step (a1-5),
(a1-6) inserting and securing a catheter of a reservoir at the externally-facing end of the tube, and sealing a tank of the reservoir with a material pierceable by a drug injection needle.

5. The method of treating according to claim 1, wherein in the (b1) step, the drug is automatically injected using a wireless injector.

6. The method of treating according to claim 1, wherein in the step (b1), the drug is continuously injected using a micropump.

7. The method of treating according to claim 1, wherein in the (b1) step, the drug is manually injected by the patient.

8. The method of treating according to claim 1, wherein the intraocular disease is selected from the group consisting of glaucoma, cataract, keratitis, and anterior uveitis.

9. A method of treating an intraocular disease comprising:
(a2) a physician places an internally-facing end of a tube for drug injection into a vitreous body of a patient; and
(b2) under a guidance of the physician, remotely injecting a drug into the vitreous body through the placed tube, wherein
the step (a2) comprises
(a2-1) incising a conjunctiva and piercing a tube insertion needle into a sclera near a corneal limbus,
(a2-2) advancing a tip of the tube insertion needle into the vitreous body,
(a2-3) inserting the internally-facing end of the tube into the vitreous body from an inlet of the tip of the tube insertion needle, and
(a2-4) placing the internally-facing end of the tube into the vitreous body and pulling out the tube insertion needle.

10. The method of treating according to claim 9, wherein the step (a2) comprises, prior to the step (a2-1) or after the step (a2-4),
(a2-0) placing an implant having a magnet in an equatorial scleral pocket.

11. The method of treating according to claim 9, wherein the step (a2) comprises, after the step (a2-4),
(a2-5) incising the skin of the external canthus, guiding an externally-facing end of the tube through the subconjunctiva and subcutaneous, and exposing the externally-facing end under the incised skin.

12. The method of treating according to claim 11, wherein the step (a2) comprises, after the step (a2-5),
(a2-6) inserting and securing a catheter of a reservoir at the externally-facing end of the tube, and sealing a tank of the reservoir with a material pierceable by a drug injection needle.

13. The method of treating according to claim 9, wherein in the (b2) step, the drug is automatically injected using a wireless injector.

14. The method of treating according to claim 9, wherein in the step (b2), the drug is continuously injected using a micropump.

15. The method of treating according to claim 9, wherein in the (b1) step, the drug is manually injected by the patient.

16. The method of treating according to claim 9, wherein the intraocular disease is selected from the group consisting of glaucoma, vitreous opacity, uveitis, diabetic retinopathy, retinal vein occlusion, macular degeneration, and retinitis pigmentosa.

17. A method of treating an intraocular disease comprising:
(a3) a physician places an internally-facing end of a tube for drug injection into a choroid of a patient; and
(b3) under a guidance of the physician, remotely injecting a drug into the choroid through the placed tube, wherein
the step (a3) comprises
(a3-1) incising a conjunctiva, and piercing a tube insertion needle between the conjunctiva and a sclera,
(a3-2) advancing a tip of the tube insertion needle to a vicinity of an optic disc along a surface of the sclera, placing a lens over a cornea so that a fundus can be observed, and observing the tip of the tube insertion needle that can be observed as a white raised portion of the sclera while pressing the sclera using the tip of the tube insertion needle and observing the fundus through the lens,
(a3-3) moving the tip of the tube insertion needle to determine an appropriate insertion position of the tube in the vicinity of the optic disc,
(a3-4) piercing the tip of the tube insertion needle diagonally into the sclera, advancing the tip of the tube insertion needle into the choroid, and inserting the internally-facing end of the tube into the choroid through an inlet of the tip of the tube insertion needle,
(a3-5) placing the internally-facing end of the tube into the choroid and pulling out the tube insertion needle, and
(a3-6) incising the skin of the external canthus, guiding the externally-facing end of the tube through the subconjunctiva and subcutaneous, and exposing the externally-facing end under the incised skin.

18. The method of treating according to claim 17, wherein the step (a3) comprises, prior to the step (a3-1) or after the step (a3-6),
(a3-0) placing an implant having a magnet in a posterior polar scleral pocket.

19. The method of treating according to claim 17, wherein the step (a3) comprises, after the step (a3-6),
(a3-7) inserting and securing a catheter of a reservoir at the externally-facing end of the tube, and sealing a tank of the reservoir with a material pierceable by a drug injection needle.

20. The method of treatment according to claim 17, wherein in the (b3) step, the drug is automatically injected using a wireless injector.

21. The method of treatment according to claim 17, wherein in the step (b3), the drug is continuously injected using a micropump.

22. The method of treating according to claim 17, wherein in the (b1) step, the drug is manually injected by the patient.

23. The method of claim 17, wherein the intraocular disease is selected from the group consisting of glaucoma, diabetic retinopathy, macular degeneration, retinal vein occlusion, uveitis, retinochoroidal degeneration, and scleritis.
